(19) United States
(12) Patent Application Publication
(10) Pub. No.: US 2008/0301882 A1
(43) Pub. Date: Dec. 11, 2008

(54) Combining BioPolishing and Bleach Clean-up

(75) Inventors: Harm Albertus Kuilderd, Beijing (CN); Guifang Wu, Beijing (CN); Haijing Li, Beijing (CN); Quan Zhou, Beijing (CN)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/136,841

(22) Filed: Jun. 11, 2008

Related U.S. Application Data
(60) Provisional application No. 60/943,861, filed on Jun. 14, 2007.

(30) Foreign Application Priority Data
Jun. 11, 2007 (EP) 07109969.1

(51) Int. Cl. D06M 16/00; C12N 9/08; C12N 9/42

(52) U.S. Cl. 8/115.51; 435/192; 435/209

(57) ABSTRACT

The present invention provides methods and compositions for treating textile, wherein the textile is treated by a system for removing hydrogen peroxide and an enzyme system for bio-polishing in one step to achieve the biopolishing and bleach clean up effect. The present invention further provides a one step process to achieve biopolishing.

### CROSS-REFERENCE TO RELATED APPLICATIONS

This application claims priority or the benefit under 35 U.S.C. 119 of European application no. EP 07109969.1 filed Jun. 11, 2007 and U.S. provisional application No. 60/943,861 filed Jun. 14, 2007, the contents of which are fully incorporated herein by reference.

### CROSS-REFERENCE TO A SEQUENCE LISTING

The present application contains information in the form of a sequence listing, which is submitted on a data carrier accompanying this application. The contents of the data carrier are fully incorporated herein by reference.

### FIELD OF THE INVENTION

The present invention relates to methods and compositions for treating textile, and more particularly, to methods and compositions for one-step combined biopolishing and bleach clean-up, comprising treating textile with a system for removing hydrogen peroxide and an enzyme system for bio-polishing.

### BACKGROUND OF THE INVENTION

Usually, there is a process to remove the residual hydrogen peroxide after bleaching. There are three kinds of way to do it. One is washing with water several times, one is treating the bath with reducing agents and the other is with the use of catalase. The using of catalase has become more and more popular. The preparation process, which may involve desizing (for woven goods), scouring, bleaching and bleach clean up, produces a textile suitable for dyeing. Bio-polishing is done before or after dyeing. Up to now, the Bleach Clean up, BioPolishing and dyeing are done in separate steps.

A. BioPolishing: Bio-Polishing is a specific treatment of the yarn surface which improves fabric quality with respect to handle and appearance. The most important effects of BioPolishing can be characterized by less fuzz and pilling, increased gloss/luster, improved fabric handle, increased durable softness and improved water absorbency. Bio-Polishing usually takes place in the wet processing of the manufacture of knitted and woven fabrics. Wet processing comprises such steps as, e.g., desizing, scouring, bleaching, washing, dying/printing and finishing.

B. Bleach Clean-up: The purpose of bleaching is to completely remove colored impurities, improve absorbency, and achieve adequate whiteness and dyeability. In order to obtain clear, pure tones on a variety of fibers (particularly on natural fibers), it is necessary to bleach them prior to dyeing. This enables one to obtain a good ground fiber. From an ecological and technical point of view, hydrogen peroxide, its derivatives and addition products that release peroxide are generally used for the bleaching process. However, generally, excess peroxide product remains on the fiber and when this occurs it can interfere with and have an adverse affect on subsequent dyeings with anionic dyes, for example, reactive dyes where the dye is in part or totally destroyed. Destroying excess H₂O₂ after bleaching is referred to as the Bleach Clean-up process. This can be done by applying a catalase to the substrate. In order to obtain consistent, high quality results with commercial quantities of textiles, the Bleach Clean-up and BioPolishing steps are usually performed separately because it is very difficult to combine the enzymatic processes due to processing constraints related to pH.

C. Dyeing: Dyeing of textiles is often considered to be the most important and expensive single step in the manufacturing of textile fabrics and garments. The major classes of dyes are azo (mono-, di-, tri-, etc.), carbonyl (anthraquinone and indigo derivatives), cyanine, di- and triphenylmethane and phthalocyanine. All these dyes contain chromophore groups, which give rise to color. These chemical structures constitute several cellulosic dye classes, i.e., vat, sulfur, azoic, direct, and reactive dyes as defined in the Color Index. Three of these dye types involve an oxidation/reduction mechanism, i.e., vat, sulfur and azoic dyes. The purpose of the oxidation/reduction step in these dyeing procedures is to change the dyestuff between an insoluble and a soluble form.

Processing and dyeing procedures are performed in either a batch or continuous mode, with the fabric being contacted by the liquid processing stream in open width or rope form. In continuous methods, a saturator is used to apply chemicals to the fabric, after which the fabric is heated in a chamber where the chemical reaction takes place. A washing section then prepares the fabric for the next processing step. Batch processing generally takes place in one processing bath whereby the fabric is circulated through the bath. After a reaction period, the chemicals are drained, fabric rinsed and the next chemical is applied. Discontinuous pad-batch processing involves a continuous application of processing chemical followed by a dwell period, which, in the case of cold pad-batch, might be one or more days.

Regardless of whether batch, continuous, or discontinuous pad-batch methods are used, bleach clean up, biopolishing and dyeing steps have not heretofore been compatible, due to the broad variation of conditions present in each of the steps. Consequently, it has been necessary to rinse or otherwise treat the fabric or to replace the treating solutions between bleach clean up, biopolishing and dyeing. Thus, there is a need in the art for harmonization of these three steps so that they can be performed in a single bath, whether simultaneously or sequentially, so as to shorten processing time, conserve materials, and reduce the waste stream.

### SUMMARY OF THE INVENTION

Therefore, the present invention provides a one-step process for combined biopolishing and beach clean up of treating textile, comprising treating the textile with (i) a system for removing hydrogen peroxide, and (ii) an enzyme system for bio-polishing, wherein the system for removing hydrogen peroxide and the enzyme system for bio-polishing are added simultaneously or sequentially to a single solution.

The process of biopolishing and beach clean up can be further combined with dyeing step and performed in a single solution. A process for combined bleach clean up, biopolishing and dyeing of treating textile, comprising (i) adding into the aqueous solution, an enzyme system for removing hydrogen peroxide, (ii) adding an enzyme system for bio-polishing, wherein steps (i) and (ii) take place simultaneously or sequentially in a single solution containing the textile, and (iii) supplementing the aqueous solution with one or more dyestuff and incubating the textile for a sufficient time to achieve dyed textile.

Preferably, the dyestuff is reactive dye. Preferably, the system for removing hydrogen peroxide is the enzyme system comprises catalase, and the enzyme system for bio-polishing comprises cellulase and/or protease.

Preferably, the system for removing hydrogen peroxide is chemical reducing agent, and the enzyme system for bio-polishing is cellulase and/or protease.

Preferably, the textile is cellulose-containing or cellulosic fabrics, such as cotton, viscose, rayon, ramie, linen, lyocell, or mixtures thereof, or mixtures of any of these fibers together with synthetic fibers or other natural fibers.

Preferably, the cellulase is derived from a species of Aspergillus, Bacillus, Fusarium, Geotricum, Humicola, Irpex, Myceliophthora, Penicillium, Phanerochaete, Schizophyllum, Scytalidium, Thermomyces, Trichoderma, or Thielavia, and more preferably the cellulase is derived from Aspergillus aculeatus, Aspergillus niger, Humicola insolens, Myceliophthora thermophila, Thielavia terrestis, Trichoderma harzianum, and Trichoderma reesei.

Preferably, the catalase is derived from bacteria such as Bacillus, Pseudomonas or Streptomyces strain; yeast such as Candida, Kluyveromyces, Pichia, Saccharomyces, Schizosaccharomyces or Yarrowia; fungi such as Acremonium, Aspergillus, Aureobasidium, Bjerkandera, Ceriporopsis, Coprinus, Coriolus, Cryptococcus, Filibasidium, Fusarium, Humicola, Magnaporthe, Mucor, Myceliphthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes or Trichoderma strain; or animals such as pig liver, beef lever. The present invention further provides an enzyme composition comprising (i) a system for removing hydrogen peroxide, and (ii) an enzyme system for bio-polishing. The system for removing hydrogen peroxide can be catalase, or chemical reducing agent. The chemical reducing agent is preferably sodium hyposulphate, while the enzyme system for biopolishing is preferably cellulase.

When biopolishing and bleach clean up take place simultaneously or sequentially in the same treating solution, compared to traditional processes involving separated biopolishing and bleach clean up, a pH adjusting step and temperature adjusting step are avoided in the present invention. Another advantage of the invention is that process time is saved/reduced as biopolishing and bleach clean up may be carried out simultaneously. The methods and compositions of the present invention of combining bio-polishing and bleaching clean-up in one step thus save time, energy and water. Furthermore, when dyeing step is combined with bleach clean up and biopolishing in the same treating solution, it makes the process even more efficient.

### DETAILED DESCRIPTION OF THE INVENTION

### Textiles

In the context of the invention the term “textile” refers to fabrics.

Fabric can be constructed from fibers by weaving, knitting or non-woven operations. Weaving and knitting require yarn as the input whereas the non-woven fabric is the result of random bonding of fibers (paper can be thought of as non-woven). In the present context, the term “fabric” is also intended to include fibers and other types of processed fabrics.

Woven fabric is constructed by weaving “filling” or weft yarns between wrap yarns stretched in the longitudinal direction on the loom. The wrap yarns must be sized before weaving in order to lubricate and protect them from abrasion at the high speed insertion of the filling yarns during weaving. The filling yarn can be woven through the warp yarns in a “over one-under the next” fashion (plain weave) or by “over one-under two” (twill) or any other myriad of permutations. Strength, texture and pattern are related not only to the type/quality of the yarn but also the type of weave. Generally, dresses, shirts, pants, sheeting's, towels, draperies, etc. are produced from woven fabric.

Knitting is forming a fabric by joining together interlocking loops of yarn. As opposed to weaving which is constructed from two types of yarn and has many “ends”, knitted fabric is produced from a single continuous strand of yarn. As with weaving, there are many different ways to loop yarn together and the final fabric properties are dependent both upon the yarn and the type of knit. Underwear, sweaters, socks, sport shirts, sweat shirts, etc. are derived from knit fabrics.

Non-woven fabrics are sheets of fabric made by bonding and/or interlocking fibers and filaments by mechanical, thermal, chemical or solvent mediated processes. The resultant fabric can be in the form of web-like structures, laminates or films. Typical examples are towels, wipes, surgical gowns, fibers for the “environmental friendly” fashion, filter media, bedding, roofing materials, backing for two-dimensional fabrics and many others.

According to the invention, the method of the invention may be applied to any fabric known in the art (woven, knitted, or non-woven). In particular the process of the present invention may be applied to cellulose-containing or cellulosic fabrics, such as cotton, viscose, rayon, ramie, linen, lyocell (e.g., Tencel, produced by Courtaulds Fibers), or mixtures thereof, or mixtures of any of these fibers together with synthetic fibers (e.g., polyester, polyamid, nylon) or other natural fibers such as wool and silk, such as viscose/cotton blends, lyocell/cotton blends, viscose/wool blends, lyocell/wool blends, cotton/wool blends; flax (linen), ramie and other fabrics based on cellulose fibers, including all blends of cellulosic fibers with other fibers such as wool, polyamide, acrylic and polyester fibers, e.g., viscose/cotton/polyester blends, wool/cotton/polyester blends, flax/cotton blends etc. The term “wool,” means any commercially useful animal hair product, for example, wool from sheep, camel, rabbit, goat, llama, and known as merino wool, Shetland wool, cashmere wool, alpaca wool, mohair, etc. and includes wool fiber and animal hair. The method of the invention can be used with wool or animal hair material in the form of top, fiber, yarn, or woven or knitted fabric. The enzymatic treatment can also be carried out on loose flock or on fibers made from wool or animal hair material. The treatment can be performed at many different stages of processing. The fabric to be bleached may be dyed or undyed. According to the invention textile may be desized, scoured and/or bleached in aqueous medium in the presence of a fatty acid oxidizing enzyme.

### Enzyme Composition

As used in the present invention, an enzyme composition comprises a bio-polishing enzyme system and a hydrogen peroxide removing system, which may be formulated as a liquid (e.g., aqueous), a solid, a gel, a paste or a dry product formulation. Accordingly, the enzyme composition may comprise one or more bio-polishing enzymes, and one or more enzymes for removing hydrogen peroxide or one or more chemical reducing agents for removing hydrogen peroxide.

“Bio-polishing enzymes system” comprises one or more bio-polishing enzyme(s), such as cellullase and protease. “Hydrogen peroxide removing system” comprises one or more enzyme(s) such as catalase, and/or chemical reducing agent(s) for removing hydrogen peroxide, which may also be referred as “Bleach Clean-up system”.

### Bio-Polishing Enzymes

The process of the invention comprises cellulase and/or protease treatment of the fabric.

Cellulase is generally used in biopolishing process for textile like cellulosic fabrics, and cellulosic fabrics/synthetic fiber blends, while protease is used in shrinkage-resistance process for natural protein fibers such as wool and silk. Preferably, cellulase together with protease can be used in biopolishing process of blends of cellulosic fibers with other fibers, such as cellulosic fabrics/wool blends, cellulosic fabrics/synthetic fibers/wool blends and etc.

### Cellulase

The term “cellulase” denotes an enzyme that contributes to the hydrolysis of cellulose, such a cellobiohydrolase (abbreviated as “CBH”, Enzyme Nomenclature E.C. 3.2.1.91), an endoglucanase (hereinafter abbreviated as “EG”, E.C. 3.2.1.4), or a beta-glucosidase (abbreviated as “BG”, E.C. 3.2.1.21). Cellulases are classified in a series of enzyme families encompassing endo- and exo-activities as well as cellobiose hydrolyzing capability. The cellulase used in practicing the present invention may be derived from microorganisms which are known to be capable of producing cellulolytic enzymes, such as, e.g., species of Aspergillus, Bacillus, Fusarium, Geotricum, Humicola, Irpex, Myceliophthora, Penicillium, Phanerochaete, Schizophyllum, Scytalidium, Thermomyces, Thielavia, or Trichoderma, particularly Fusarium oxysporum, Humicola insolens, or Trichoderma reesei. Non-limiting examples of suitable cellulases are disclosed in U.S. Pat. No. 4,435,307; European Patent Application No. 0 495 257; PCT Patent Application No. WO 91/17244, WO 91/17243, WO 98/12307; and European Patent Application No. 0271 004.

The cellulases used in this invention can be monocomponent or multi-components. Monocomponent, i.e., a cellulase which is essentially free from other proteins, in particular other cellulases. Monocomponent enzymes can be prepared economically by recombinant DNA technology, i.e., they can be produced by cloning of a DNA sequence encoding the monocomponent, subsequently transforming a suitable host cell with the DNA sequence and expressing the component in the host. Cellulases of multi-components contain more than one cellulase. Cellulases of multi-components can be blends of two or more cellulases, or can be derived from wild type strain.

The DNA sequence coding for a useful cellulase may for instance be isolated by screening a cDNA library of the microorganism in question and selecting for clones expressing the appropriate enzyme activity (i.e., cellulase activity).

A DNA sequence coding for a homologous enzyme, i.e., an analogous DNA sequence, may be obtainable from other microorganisms. For instance, the DNA sequence may be derived by similarly screening a cDNA library of another fungus, such as a strain of an Aspergillus sp., in particular a strain of A. aculeatus or A. niger, a strain of Trichoderma sp., in particular a strain of T. harzianum, T. koningi, T. longibrachiatum, T. reesei, or T. viride, or a strain of an Agaricus sp., a Neocallimastix sp., a Penicillium sp., a Phanerochaete sp., or a Piromyces sp.

Preferably, the cellulase is derived from or producible by a strain of Fusarium, Myceliophthora, Scytalidium (f. Humicola), more preferably derived from or producible by Fusarium oxysporum, Myceliophthora themophila, or Scytaidium thermophilum (f. Humicola insolens), most preferably from Fusarium oxysporum, DSM 2672, Humicola insolens, DSM 1800, or Myceliophthora themophila, CBS 117.65.

In one embodiment of the invention, the cellulase is an endoglucanase, preferably cellulase from family 45 EG, such as the amino acid sequence of the Thielavia terrestis endoglucanase shown in SEQ ID NO: 1 (as described in WO 96/29397) or is an analogue of said endoglucanase which is at least 60% homologous with the sequence shown in SEQ ID NO: 1, reacts with an antibody raised against said endoglucanase, and/or is encoded by a DNA sequence which hybridizes with the DNA sequence encoding said endoglucanase. In another embodiment of the invention, the cellulase is an endoglucanase comprising the amino acid sequence of the Humicola insolens endoglucanase shown in SEQ ID NO: 2 (as described in WO 91/17243) or is an analogue of said endoglucanase which is at least 60% homologous with the sequence shown in SEQ ID NO: 2, reacts with an antibody raised against said endoglucanase, and/or is encoded by a DNA sequence which hybridizes with the DNA sequence encoding said endoglucanase. In a further embodiment of the invention, the cellulases used in the invention are commercially available multi-components cellulase enzyme product such as Cellusoft L, Cellish L (Novozymes A/S, Denmark), Primafast 100, Primafast 200 (Genencor International Inc.), Rocksoft ACE (Dyadic), and Youteer 800 (Youteer Co. Ltd, China).

The host cell which is transformed with the DNA sequence is preferably a eukaryotic cell, in particular a fungal cell such as a yeast or filamentous fungal cell. In particular, the cell may belong to a species of Aspergillus or Trichoderma, most preferably Aspergillus oryzae or Aspergillus niger. Fungal cells may be transformed by a process involving protoplast formation and transformation of the protoplast followed by regeneration of the cell wall in a manner known per se. The use of Aspergillus as a host microorganism is described in EP 238 023 (Novo Nordisk A/S), the contents of which are hereby incorporated by reference. The host cell may also be a yeast cell, e.g., a strain of Saccharomyces, in particular Saccharomyces cerevisiae, Saccharomyces kluyveri or Saccharomyces uvarum, a strain of Schizosaccharomyces sp., such as Schizosaccharomyces pombe, a strain of Hansenula sp., Pichia sp., Yarrowia sp. such as Yarrowia lipolytica, or Kluyveromyces sp. such as Kluyveromyces lactis.

In the context, an analogue of the proteins comprises “variant proteins”. In some preferred embodiments, variants proteins differ from a parent protein, e.g., a wild-type protein, and one another by a small number of amino acid residues. In the present context, the term “homologous” or “homologous sequence” is intended to indicate an amino acid sequence differing from another protein, by one or more amino acid residues, preferably 1, 2, 3, 4, 5, 10, 15, 20, 30, 40, 50 or more amino acid residues. For example, in some embodiments, variant proteins have one to ten differences from the parent protein. The homologous sequence may be one resulting from modification of an amino acid sequence shown in these listings, e.g., involving substitution of one or more amino acid residues at one or more different sites in the amino acid sequence, deletion of one or more amino acid residues at either or both ends of the enzyme or at one or more sites in the amino acid sequence, or insertion of one or more amino add residues at one or more sites in the amino acid sequence.

However, as will be apparent to the skilled person, amino acid changes are preferably of a minor nature, that is conservative amino add substitutions that do not significantly affect the folding or activity of the protein, small deletions, typically of one to about 30 amino adds; small amino- or carboxyl-terminal extensions, such as an amino-terminal methionine residue, a small linker peptide of up to about 20-25 residues, or a small extension that facilitates purification, such as a poly-histidine tract, an antigenic epitope or a binding domain. See in general Ford et al., Protein Expression and Purification 2: 95-107, 1991. Examples of conservative substitutions are within the group of basic amino acids (such as arginine, lysine, histidine), acidic amino acids (such as glutamic acid and aspartic acid), polar amino acids (such as glutamine and asparagine), hydrophobic amino acids (such as leucine, isoleucine, valine), aromatic amino acids (such as phenylalanine, tryptophan, tyrosine) and small amino acids (such as glycine, alanine, serine, threonine, methionine).

The modification of the amino acid sequence may suitably be performed by modifying the DNA sequence encoding the enzyme, e.g., by site-directed or by random mutagenesis or a combination of these techniques in accordance with well-known procedures. Alternatively, the homologous sequence may be one of an enzyme derived from another origin than the cellulases corresponding to the amino acid sequences shown in each of the sequence listings shown hereinafter, respectively. Thus, “homologue” may, e.g., indicate a polypeptide encoded by DNA which hybridizes to the same probe as the DNA coding for the cellulase with the amino acid sequence in question under certain specified conditions (such as presoaking in 5×SSC and prehybridising for 1 h at ˜40° C. in a solution of 20% formamide, 5×Denhardt's solution, 50 mM sodium phosphate, pH 6.8, and 50 mg of denatured sonicated calf thymus DNA, followed by hybridization in the same solution supplemented with 100 mM ATP for 18 h at ˜40° C.). The homologous sequence will normally exhibit a degree of homology (in terms of identity) of at least 50%, such as at least 60%, 65%, 70%, 75%, 80%, 85%, 90% or even 95% with the amino acid sequences shown in each of the sequence listings shown hereinafter, respectively.

The homology referred to above is determined as the degree of identity between the two sequences indicating a derivation of the first sequence from the second. The homology may suitably be determined by means of computer programs known in the art such as GAP provided in the GCG program package (Needleman and Wunsch, Journal of Molecular Biology, 48: 443-453, 1970).

### Proteases

Any protease suitable for use in the present invention may be employed.

Suitable proteases include those of animal, vegetable or microbial origin, preferably of microbial origin. Preferably, the protease may be a serine protease or a metalloprotease, more preferably, an alkaline microbial protease or a trypsin-like protease. Examples of proteases include aminopeptidases, including prolyl aminopeptidase (3.4.11.5), X-pro aminopeptidase (3.4.11.9), bacterial leucyl aminopeptidase (3.4.11.10), thermophilic aminopeptidase (3.4.11.12), lysyl aminopeptidase (3.4.11.15), tryptophanyl aminopeptidase (3.4.11.17), and methionyl aminopeptidase (3.4.11.18); serine endopeptidases, including chymotrypsin (3.4.21.1), trypsin (3.4.21.4), cucumisin (3.4.21.25), brachyurin (3.4.21.32), cerevisin (3.4.21.48) and subtilisin (3.4.21.62); cysteine endopeptidases, including papain (3.4.22.2), ficain (3.4.22.3), chymopapain (3.4.22.6), asclepain (3.4.22.7), actinidain (3.4.22.14), caricain (3.4.22.30) and ananain (3.4.22.31); aspartic endopeptidases, including pepsin A (3.4.23.1), Aspergillopepsin I (3.4.23.18), Penicillopepsin (3.4.23.20) and Saccharopepsin (3.4.23.25); and metalloendopeptidases, including Bacillolysin (3.4.24.28).

Commercially available proteases include Alcalase, Savinase, Primase, Duralase, Esperase, Kannase, and Durazym (available from Novozymes A/S), Maxatase, Maxacal, Maxapem, Properase, Purafect, Purafect OxP, FN2, FN3 and FN4 (available from Genencor International Inc.).

Also useful in the present invention are protease variants, such as those disclosed in EP 130,756 (Genentech), EP 214,435 (Henkel), WO 87/04461 (Amgen), WO 87/05050 (Genex), EP 251.446 (Genencor), EP 260.105 (Genencor), Thomas et al., 1985, Nature 318: 375-376, Thomas et al., 1987, J. Mol. Biol. 193: 803-813, Russel et al., 1987, Nature 328: 496-500, WO 88/08028 (Genex), WO 88/08033 (Amgen), WO 89/06279 (Novozymes A/S), WO 91/00345 (Novozymes A/S), EP 525 610 (Solvay) and WO 94/02618 (Gist-Brocades N.V.). The activity of proteases can be determined as described in “Methods of Enzymatic Analysis”, third edition, 1984, Verlag Chemie, Weinheim, vol. 5.

### Bleach Clean-Up System

The Bleach Clean-up system can be Bleach Clean-up enzyme and/or chemical reducing agent to remove hydrogen peroxide in solution.

### Bleach Clean-Up Enzymes

Bleach clean-up enzymes refer to enzymes which can catalyze the conversion of hydrogen peroxide into water and oxygen, such as catalase (EC 1.11.1.6). Preferred catalases that are suitable for use in a process according to the invention are catalases that are derived from bacteria such as Bacillus, Pseudomonas or Streptomyces strain; yeast such as Candida, Kluyveromyces, Pichia, Saccharomyces, Schizosaccharomyces or Yarrowia; fungi such as Acremonium, Aureobasidium, Aspergillus, Bjerkandera, Ceriporiopsis, Coprinus, Coriolus, Cryptococcus, Filibasidium, Fusarium, Humicola, Magnaporthe, Mucor, Myceliphthora, Neocallimastix, Neurospora, Paecilomyces, Penicillium, Phanerochaete, Phlebia, Piromyces, Pleurotus, Schizophyllum, Scytalidium, Talaromyces, Thermoascus, Thielavia, Tolypocladium, Trametes or Trichoderma strain; or animals such as pig liver, beef lever. Non-limiting examples of suitable catalases are disclosed in WO 92/17571, CN 1563373, US 2003100112-A1, EP 1336659-A, US 2003/074697, U.S. Pat. No. 6,201,1671, U.S. Pat. No. 6,022,721, EP 931831-A, JP 11046760-A, WO 93/17721, WO 93/09219, JP 1086879-A and/or JP 63003788-A. Non-limiting examples are T 100; Oxy-Gone 400 (GCI); Fermcolase 1000 (Mitsubishi Gas Chemical) or Thermocatalase CTL 200 or JH CT 1800 (Mitsubishi Gas Chemical).

In one embodiment of the present invention, the catalase used is derived from Scytalidium thermophilum with SEQ ID NO: 3 (as described in U.S. Pat. No. 5,646,025), or analogue or particularly variant proteins of it.

Depending on the activity of the catalase and the pH of the liquor used to apply the catalase, preferably the amount of catalase used is from 0.001 to 1 g/l, especially about 5 g/l of liquor used to apply the catalase.

### Chemical Reducing Agent

Chemical reducing system refers to any chemical reducing agent(s) for removing hydrogen peroxide by catalyzing the conversion of hydrogen peroxide into water and oxygen. Preferably, the reducing agent can be those widely used in textile industry, like sodium thiosulphate, sodium bisulphite, sodium hydrosulphite and sodium hyposulphate etc.

### Dyestuff

In practicing the present invention, any dyestuff may be used that is compatible with the conditions used for biopolishing.

Conventional dyeing stuffs, comprising one or more of reactive dyes, such as, e.g., C. I. Reactive Red 1, 3, 6, 17, 120, 194, Blue 4, 19, 171 and 182, Black 5, Violet 5; vat dyes, such as, e.g., C. I. Vat Yellow 28, Orange 11 and 15, Blue 6, 16 and 20, Green 1 and 3, 8, Brown 1, Black 9, 27; direct dyes, such as, C. I. Direct Red 81, Yellow 11 and 28, Orange 39, Red 76, Blue 78, 86, 106, 107 and 108, Black 22; sulfur dyes, such as, e.g., C. I. Sulfur Black 1 and 11, Brown 1, Red 10; and azoic dyes, such as, e.g., C. I. Coupling Components 5 and 13 in combination with C. I. Azoic Diazo Components 44 and 45. Such dyes are well known in the art and are described, e.g., in Shore, ed., Cellulosic Dyeing, Society of Dyers and Colorists, Alden Press, 1995; and in Color Index, Society of Dyers and Colorists and American Association of Textile Chemists and Colorists, Vols. 1-8 Supplements, 1977-1988.

Additional Components

In some embodiments of the invention, the aqueous solution or wash liquor further comprises other components, including without limitation other enzymes, as well as surfactants, stabilizer, wetting agent, dispersing agents, antifoaming agents, lubricants, builder systems, and the like, or a mixture thereof, that enhance the scouring and/or bleaching processes and/or provide superior effects related to, e.g., strength, resistance to pilling, water absorbency, and dyeability.

The enzymes may be isolated from their cell of origin or may be recombinantly produced, and may be chemically or genetically modified. It will be understood that the amount of enzymatic activity units for each additional enzyme to be used in the methods of the present invention in conjunction with a particular bio-polishing enzyme can be easily determined using conventional assays.

Surfactants suitable for use in practicing the present invention include, without limitation, nonionic, anionic; cationic; and zwitterionic surfactants; which are typically present at a concentration of between about 0.2% to about 15% by weight, preferably from about 1% to about 10% by weight. Anionic surfactants include, without limitation, linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid, and soap. Non-ionic surfactants include, without limitation, alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, and N-acyl N-alkyl derivatives of glucosamine (“glucamides”).

Builder systems include, without limitation, aluminosilicates, silicates, polycarboxylates and fatty acids, materials such as ethylenediamine tetraacetate, and metal ion sequestrants such as aminopolyphosphonates, particularly ethylenediamine tetramethylene phosphonic acid and diethylene triamine pentamethylenephosphonic acid, which are included at a concentration of between about 5% to 80% by weight, preferably between about 5% and about 30% by weight.

Antifoam agents include without limitation silicones (U.S. Pat. No. 3,933,672; DC-544 (Dow Corning), which are typically included at a concentration of between about 0.01% and about 1% by weight.

The compositions may also contain soil-suspending agents, soil-releasing agents, optical brighteners, abrasives, and/or bactericides. All of such further components suitable for textile use are well know in the art.

### Process for Single Bath Bleach Clean Up and Biopolishing

The process of the present invention of one-step combined biopolishing and bleach clean up takes place after bleaching step for treating textile.

The manner in which the aqueous solution containing the enzyme system for BioPolishing and Bleach Clean-up system is contacted with the textile will depend upon whether the processing regime is continuous, discontinuous pad-batch or batch. For example, for continuous or discontinuous pad-batch processing, the aqueous enzyme solution is preferably contained in a saturator bath and is applied continuously to the cotton-containing fabric as it travels through the bath, during which process the cotton-containing fabric typically absorbs the processing liquor at an amount of 0.5-1.5 times its weight. In batch operations, the cotton-containing fabric is exposed to the enzyme solution for a period ranging from about 5 minutes to 24 hours at a liquor-to-fabric ratio of 4:1-50:1.

The aqueous solution or wash liquor typically has a pH of between about 4 and about 11. Preferably, the pH of the treating composition is between about 5 and about 10, preferably between about 5 to about 9, and most preferably about 6 to about 7.

In one embodiment, the single-bath method for treating cotton-containing fabric is carried out at a pH below 9, more preferably, below 8, and even more preferably below 7.

The temperature at which the combined Bleach Clean-up and BioPolishing processes are carried out will depend on the process used. In the case of cold pad batch process, the temperature for the combined bleaching clean-up and bio-polishing processes is preferably between about 15° C. and about 65° C., and most preferably between about 25° C. and about 60° C. For continuous and other batch processes, the temperature for carrying out combined bleaching clean-up and bio-polishing processes is preferably between about 35° C. and about 75° C., and most preferably between about 45° C. and about 65° C.

The enzyme for biopolishing is generally added in an amount which is effective to generate enough biopolishing effect of the textile material. The enzyme(s) may preferably be dosed in an amount of 0.01 mg protein/l to 1 g protein/l of the total liquor, more preferably from 0.1 mg protein/l to 300 mg protein/l, most preferably from 1 mg protein/l to 150 mg protein/l.

The enzyme for bleach clean up is generally added in an amount which is effective to remove hydrogen peroxide. The enzyme(s) may preferably be dosed in an amount about from 0.001 mg protein/l to 1 g protein/l of the total liquor, preferably 0.01 mg protein/l to 100 mg protein/l, most preferably 0.15 mg protein/l to about 1 mg protein/l.

The chemical reductive agent for bleach clean up may be dosed in an amount of from about 0.01 g/l to about 10 g/l of the total liquor, more preferably, from about 0.1 g/l to about 3 g/l, most preferably, from about 0.5 g/l to about 1 g/l.

It will be understood that the optimum dosage and concentration of the enzymes, bleach compounds, bleach stabilizers, the volume of the aqueous solution or wash liquor, and the pH and temperature will vary, depending on: (i) the nature of the fiber, i.e., crude fiber, yarn, or textile; (ii) the particular enzyme(s) used, and the specific activity of the enzyme; (iii) the conditions of temperature, pH, time, etc., at which the processing occurs; (iv) the presence of other components in the wash liquor; and (v) the type of processing regime used, i.e., continuous, discontinuous pad-batch, or batch. The optimization of the process conditions can be determined using routine experimentation, such as, by establishing a matrix of conditions and testing different points in the matrix. For example, the amount of enzyme, the temperature at which the contacting occurs, and the total time of processing can be varied, after which the resulting cellulosic materials or textile is evaluated for (a) pilling result; and (b) residual H₂O₂ ppm.

Further, according to the present invention, bleach clean up, biopolishing and dyeing steps can be achieved in a single-bath. In one embodiment, (i) adding catalase and cellulase for bleach clean up and biopolishing, a first incubation is performed for a sufficient time to remove the residual hydrogen peroxide, (ii) the wash liquor is then supplemented with dyestuffs and a second incubation is performed for a sufficient time and under appropriate conditions to achieve effective dyeing. Preferably, after adding catalase and cellulase the incubation time for step (i) is no less than 5 minutes, so most of the hydrogen peroxide has been removed. More preferably, the incubation time for step (i) is no less than 10 minutes, even more preferably no less than 15 minutes and no more than 2 hours. Most preferably, the incubation time for step (i) is 10-20 minutes, so as to remove almost all of the hydrogen peroxide and shorten the whole process time.

It will be understood that before or during step (ii) of dyeing, it further comprise adjusting one or more properties of the composition of the wash liquor, such as ionic strength by adding salt. And the conditions of incubation in dyeing step may also differ with respect to temperature, agitation, pH, time, and the like. The performances of biopolishing with enzymes still continue during incubation of dyeing process, so that the whole process time will be shortened.

Preferably, the dyestuff used in present invention is reactive dye, which works well with cellulase disclosed herein.

The salt added before or after the adding of dyestuff preferably is NaCl or Na₂SO₄ which take control of the uptake of the dyestuff on the fiber. The concentration of salt in the wash liquor depends on the concentration of dyestuff and what kind of dyestuff used. Normally, the higher concentration of dyestuff is required, the higher salt concentration is needed. Typically, the dyestuff in the wash liquor is 0.001-15%, preferably 0.001-9% of the weight of fabric. The concentration of salt (e.g., Na₂SO₄) is 10-100 g/l.

The wash liquor in dyeing step preferably has a pH between about 5 and about 8, most preferably between about 6 and about 7.

The temperature at which the subsequent dyeing is carried out may be between about 30° C. and about 100° C., preferably between about 40° C. and about 90° C., and most preferably between about 40° C. and about 80° C.

It will be understood that in most cases, the pH and temperature in bleach clean up and biopolishing step are suitable for the dyeing step, especially for reactive dye. So, there is no need to adjust pH and temperature during dyeing.

The following are intended as non-limiting illustrations of the present invention.

### EXAMPLES

### Materials & Methods

### Enzymes

Catalase A: Catalase with SEQ ID NO: 3

Catalase T100 (Genencor international Inc)

Catalase ASC 200 (Mitsubishi)

Neutral Cellulase B with SEQ ID NO: 2

Neutral Cellulase A with SEQ ID NO: 1

Acid Cellulase C: CELLUSOFT L™ (Novozymes A/S)

Primafast Luna RL (Genencor international Inc)

IndiAge Max L (Genencor international Inc)

### Fabric

460-60 bleached Interlock Knits (Testfabrics, Inc.)

Dyed fabric (Garment bought from Carrfour)

### Dyestuff

Reactive RED BF-3B (Arugs textile auxiliary CO.LTD)
Reactive Yellow BF-3R (Arugs textile auxiliary CO.LTD)
Reactive TQ blue G (Arugs textile auxiliary CO.LTD)

### Buffer

2.716 g of potassium dihydrogen phosphate and 0.201 g sodium hydroxide are dissolved in 1 L de-ionized water
1 g/l NAAC adjust with HAC to pH 5

### Methods

### Catalase Activity (KCIU Assay)

When used according to the present invention the activity of catalase may be measured in KCIU. One CIU will decompose one micro-mole of H₂O₂ per minute at pH 7.0, 25° C. while the H₂O₂ concentration decreases from 10.3 to 9.2 micro-moles per ml reaction mixture.

Catalases catalyze the first order reaction:

2H₂O₂→2H₂O+O₂

The degradation of hydrogen peroxide is monitored using spectrophotometry at 240 nm. The length of time for a specified decrease in absorbance, at a specified H₂O₂ concentration, is a measure of the catalase activity.

Reaction

| | |
|---|---|
| Enzyme concentration | approx. 100 CIU/ml |
| Substrate Concentration | 10.3 mM H₂O₂ |
| pH | 7.0 ± 0.05 |
| Buffer | 50 mM phosphate |
| Temperature | 25° C. |

Detection

| | |
|---|---|
| Wave length | 240 nm |
| Absorbance interval | 0.450-0.400 |
| Time interval | 0.267-0.400 minutes (16-24 seconds) |

A folder describing this analytical method in more detail is available upon request to Novozymes A/S, Denmark, which folder is hereby included by reference.

### Cellulase Activity (ECU)

Cellulase samples are incubated with a carboxymethyl cellulose (CMC) substrate. Degradation of the substrate leads to a reduction in viscosity, which is measured using a vibrating-spindle viscometer. The reduction in viscosity is proportional to the endo-cellulase activity.

Endo-cellulase activity in ECU is measured relative to a Novozymes A/S enzyme standard.

Reaction conditions

| | |
|---|---|
| Temperature | 40° C. |
| pH | 7.5 |
| Substrate concentration | 3.11% (m/V) |
| Incubation time | 30 min |
| Enzyme concentration | 0.097-0.181 ECU/ml |

A folder describing this analytical method in more detail is available upon request to Novozymes A/S, Denmark, which folder is hereby included by reference.

### Cellulase Activity (EGU)

Cellulase samples are incubated with carboxymethyl cellulose (CMC) substrate. Degradation of the substrate leads to a reduction in viscosity, which is measured using a vibrating-spindle viscometer. The reduction in viscosity is proportional to the endo-glucanase activity.

Reaction Conditions

| | |
|---|---|
| Temperature | 40° C. |
| pH | 6.0 |
| Substrate concentration | 3.11% (w/v) |
| Enzyme concentration | 0.01-0.02 EGU/mL |
| Reaction time | 30 minutes |

A folder describing this analytical method in more detail is available upon request to Novozymes A/S, Denmark, which folder is hereby included by reference.

Bleach clean Up (Peroxide Test-Strip

Peroxidases transfer peroxide oxygen to an organic redox indicator. This produces a blue oxidation product. This peroxide concentration is measured semi-quantitatively by visual comparison of the reaction zone of the analytical test strip with the fields of a color scale. The hydrogen peroxide is determined by immersing the reaction zone of the analytical test strip in the measurement sample for 1 second. Allow excess liquid to run off via the long edge of strip onto an absorbent paper towel and after 15 sec. determine with which color field on the label on the color of the reaction zone coincides most exactly. Read off the corresponding result in mg/l H₂O₂ or if necessary, estimate an intermediate value.

### Pilling Test

### Martindale

The principle of the pilling test is that two specimens of a test fabric are rubbed against each other in a continuously changing pattern, which ensures that the surface fibers of the specimens are flexed in every direction. After a predetermined number of rubbing cycles, the wear resistance of the specimens is visually evaluated by comparing to EMPA photo scale standards. There are five grades of the pilling results

Note 5: No pilling

Note 4: Slight Pilling

Note 3: Moderate Pilling

Note 2: Distinct Pilling

Note 1: Heavy Pilling

½ notes are allowed

The testing method refers to GB/T 4802.2-1997

### The ICI Pilling Tester

The ICI Pilling Tester is the most applicable test to use on most types of fabric, both woven and knitted, and its test method runs in accordance with BS EN ISO 12945-1 (among other standards). The Pilling Tester consists of 2 boxes, each being lined with a metal plate supporting 3.2 mm thick buffed-finish cork jointing material. The tester features an automatic counter which stops the machine after any predetermined number of revolutions.

Assessment of the pilling standard is visual, using either the standard photographs or the Rating Scheme. The tested specimens are mounted on card and then rated against the standard photos or against an untested sample.

| | | Points to be taken into |
|---|---|---|
| | | consideration during |
| Rating | Description | assessment |
| 5 | No change | No visual change |
| 4 | Slight change | Slight surface fuzzing |
| 3 | Moderate change | Test specimen may exhibit |
| | | moderate fuzzing and/or |
| | | isolated fully formed pills |
| 2 | Significant change | Distinct fuzzing and/or |
| | | pilling |
| 1 | Severe change | Dense fuzzing and/or pilling |
| | | which covers the specimen |

### Protein Content

BCA kit (available from PIERCE) was used for detecting the protein content in the enzyme product.

### Example 1

### Combining Bio-Polishing and Bleach Clean Up Into One Process Conducted with a Laundry-Meter

A 100% 460-60 bleached interlock cotton fabric was purchased from Test Fabrics. Fabric swatches were cut to about 5 g each.

One buffer at pH 6.5 was used for this study. 2.716 g of potassium dihydrogen phosphate and 0.201 g sodium hydroxide were dissolved in 1 L de-ionized water. The process was conducted with a Laundry-meter. The beaker was filled with 100 ml buffer and two pieces of pre-cut fabric.

1) Main washing: The beaker was filled with 100 ml buffer. Some hydrogen peroxide was added to each beaker with 20 steel balls as specified. In the meanwhile, a neutral Cellulase A was dosed to a concentration and catalase A was added in then temperature was raised to 55° C. and kept for 60 min.
2) Check the residual Hydroperoxide with peroxide strip.
3) Inactivation: After the checking, add the 1 g/l Na₂CO₃ in the machine/beaker then raised the temperature to 80° C. and run for 10 min, drained.
4) Cold rinse: Filled in cold water and rinsed for 10 min
5) Spin off the water on the fabrics and tumble dryer.
6) Measure the weight loss and pilling result on the treated fabrics.

The results of the test are shown in Table 1.

TABLE 1

| H₂O₂ | Catalase A | Neutral | Residual H₂O₂ | |
|---|---|---|---|---|
| at | (mg | Cellulase A | after | Pilling |
| beginning | protein/l) | (mg protein/l) | treatment | result |
| 0 | 0 | 0 | 0 | 1.5-2 |
| 0 | 0 | 7 | 0 | 4-4.5 |
| 100 ppm | 0 | 7 | >25 ppm | 4-4.5 |
| 0 | 0.34 | 7 | 0 | 4.5 |
| 100 ppm | 0.34 | 0 | 0 | 1.5-2 |
| 100 ppm | 0.34 | 7 | 0 | 4.5 |
| 350 ppm | 0.34 | 4.2 | 0 | 4-4.5 |

As can be seen from the table, the fabrics treated in the combined process of the invention with a combination of catalase and cellulase have less pilling, and no detectable Residual H₂O₂ in the solution.

### Example 2

### Combining Bio-Polishing and Bleach Clean Up Into One Process Conducted with a Wascator

A 100% 460-60 bleached interlock cotton fabric was purchased from Test Fabrics. The weight of the fabric swatches was 1 kg.

One buffer at pH 6.5 was used for this study. 2.716 g of potassium dihydrogen phosphate and 0.201 g sodium hydroxide were dissolved in 1 L de-ionized water. The process was conducted with a wascator. 1 kg fabric was put in the wascator at the beginning of the process.

1) Main washing: The wascator was filled with 10 L water. The temperature was raised to 55° C. In the mean while, 36 g of K₂HPO₄ and 14.5 g KH₂PO₄ were added to adjust the pH to 6.5. Some hydrogen peroxide was added to the wascator as specified. A neutral cellulase was dosed to a concentration and catalase was added in and kept for 60 min.
2) Check the residual Hydroperoxide with peroxide strip after 20 min.
3) Inactivation: After main washing, drained and re-fill the wascator and added the 1 g/l Na₂CO₃ in the wascator then raised the temperature to 80° C. and run for 10 min, drained.
4) Cold rinse: Filled in cold water and rinsed for 10 min
5) Spin off the water on the fabrics and tumble dryer.
6) Measure the pilling result on the treated fabrics.

The result of the testing are shown in Table 2

TABLE 2

| H₂O₂ | Catalase A | Neutral | Residual H₂O₂ | |
|---|---|---|---|---|
| at | (mg | Cellulase A | after treatment | Pilling |
| beginning | protein/l) | (mg protein/l) | for 20 min | result |
| 100 ppm | 0.34 | 7 | 0 | 4.5 |

As can be seen from the table, the fabrics treated in the combined process of the invention with a combination of catalase A and neutral cellulase A have less pilling, and no detectable residual H₂O₂ in the solution.

### Example 3

### Combining Bio-Polishing and Bleach Clean Up Into One Process Conducted on Dyed Fabric with a Laundrymeter

A 100% dyed cotton fabric was purchased. Fabric swatches were cut to about 10 g each.

One buffer at pH 6.5 was used for this study. 2.716 g of potassium dihydrogen phosphate and 0.201 g sodium hydroxide were dissolved in 1 L de-ionized water. The process was conducted with a Laundry-meter. The beaker was filled with 100 ml buffer and one piece of pre-cut fabric.

1) Main washing: The beaker was filled with 100 ml buffer. Some hydrogen peroxide was added to each beaker with 20 steel balls as specified. In the meanwhile, a neutral Cellulase A was dosed to a concentration and catalase A was added in then temperature was raised to 55° C. and kept for 20 min.
2) Check the residual Hydroperoxide with peroxide strip.
3) Inactivation: After the checking, add the 1 g/l Na₂CO₃ in the machine/beaker then raised the temperature to 80° C. and run for 10 min, drained.
4) Cold rinse: Filled in cold water and rinsed for 10 min.
5) Spin off the water on the fabrics and tumble dryer.
6) Measure pilling result on the treated fabrics.

The results of the test are shown in Table 3.

TABLE 3

| H₂O₂ | Catalase A | Neutral | Residual H₂O₂ | |
|---|---|---|---|---|
| at | (mg | Cellulase A | after | Pilling |
| beginning | protein/l) | (mg protein/l) | treatment | result |
| 0 | 0 | 0 | 0 | 2-2.5 |
| 350 ppm | 0 | 0 | >25 ppm | 2-2.5 |
| 0 | 0 | 4.2 | 0 | 4 |
| 350 ppm | 0 | 4.2 | >25 ppm | 3.85 |
| 350 ppm | 0.34 | 4.2 | 0 | 4 |
| 0 | 0 | 21 | 0 | 4.5-5 |
| 350 ppm | 0 | 21 | >25 ppm | 4.5-5 |
| 350 ppm | 0.34 | 21 | 0 | 4.5-5 |

As can be seen from the above table, the fabrics treated in the combined process of the invention with a combination of catalase and cellulase have less pilling, and no detectable residual H₂O₂ in the solution. The present invention shows more advantageous effect than those using catalase or cellulase alone.

### Example 4

### Combining Bio-Polishing and Bleach Clean Up Into One Process with an Acid Cellulase

A 100% 460-60 bleached interlock cotton fabric was purchased from Test Fabrics. Fabric swatches were cut to about 5 g each.

One buffer at pH 5 was used for this study. 1 g of sodium acetate was dissolved in 1 L de-ionized water and adjusted the pH to 5 with acetic acid. The process was conducted with a Laundry-meter. The beaker was filled with 100 ml buffer and two pieces of pre-cut fabric.

1) Main washing: The beaker was filled with 100 ml buffer. Some hydrogen peroxide was added to each beaker with 20 steel balls as specified. In the meanwhile, an acid Cellulase C was dosed to a concentration and catalase A was added in then temperature was raised to 55° C. and kept for 60 min.
2) Check the residual Hydroperoxide with peroxide strip.
3) Inactivation: After the checking, add the 1 g/l Na₂CO₃ in the machine/beaker then raised the temperature to 80° C. and run for 10 min, drained.
4) Cold rinse: Filled in cold water and rinsed for 10 min.
5) Spin off the water on the fabrics and tumble dryer.
6) Measure the weight loss and pilling result on the treated fabrics.

The results of the test are shown in Table 4.

TABLE 4

| H₂O₂ | Catalase A | Acid | Residual H₂O₂ | |
|---|---|---|---|---|
| at | (mg | Cellulase C | after | Pilling |
| beginning | protein/l) | (mg protein/l) | treatment | result |
| 0 | 0 | 0 | 0 | 1.5-2 |
| 0 | 0 | 120 | 0 | 4-4.5 |
| 100 ppm | 0.34 | 120 | 0 | 3.5-4 |

### Example 5

### Combining Bio-Polishing and Bleach Clean Up Into One Process with Primafast Luna RL

The procedures were the same as described in Example 4 whilst the cellulase used was Primafast luna RL and the catalse used was Catalase A or Catalase T100, ASC 200 individually. The results of the test are shown in Table 5.

### Example 6

### Combining Bio-Polishing and Bleach Clean Up Into One Process with IndiAge Max L

The procedures were the same as described in Example 4 whilst the cellulase used was Primafast luna RL and the catalase used was Catalase A or Catalase T100, ASC 200 individually. The results of the test are shown in Table 5.

### Example 7

### Combining Bio-Polishing and Bleach Clean Up Into One Process with Neutral Cellulase A

The procedures were the same as described in Example 1 whilst the Catalase used was Catalase T 100 or ASC 200. The results of the test are shown in Table 5.

### Example 8

### Combining Bio-Polishing and Bleach Clean Up Into One Process with Acid Cellulase C

The procedures were the same as described in Example 4 whilst the Catalase used was Catalase T 100 or ASC 200. The results of the test are shown in Table 5.

TABLE 5

| | | | | | | Residual |
|---|---|---|---|---|---|---|
| H₂O₂ | | Dosage | | Dosage | Pilling | H₂O₂ after |
| at beginning | Cellulase | (mg protein/l) | Catalase | (mg protein/l) | result | treatment |
| / | Primafast luna | 41.88 | / | / | 3.85 | 0 |
| | RL | | | | | |
| 350 ppm | Primafast luna | 41.88 | Catalase A | 0.34 | 4-4.5 | 0 |
| | RL | | | | | |
| 350 ppm | Primafast luna | 41.88 | Catalase T | 1 | 3.85 | 0 |
| | RL | | 100 | | | |
| 350 ppm | Primafast luna | 41.88 | ASC 200 | 0.58 | 3.85 | 0 |
| | RL | | | | | |
| / | IndiAge Max L | 69 | / | / | 4.5-5 | 0 |
| 350 ppm | IndiAge Max L | 69 | Catalase A | 0.34 | 4-4.5 | 0 |
| 350 ppm | IndiAge Max L | 69 | Catalase T | 1 | 4-4.5 | 0 |
| | | | 100 | | | |
| 350 ppm | IndiAge Max L | 69 | ASC 200 | 0.58 | 4-4.5 | 0 |
| / | Neutral | 1.75 | / | / | 4-4.5 | 0 |
| | cellulase A | | | | | |
| 350 ppm | Neutral | 1.75 | Catalase T | 1 | 3.5-4 | 0 |
| | cellulase A | | 100 | | | |
| 350 ppm | Neutral | 1.75 | ASC 200 | 0.58 | 4-4.5 | 0 |
| | cellulase A | | | | | |
| / | Acid cellulase | 120 | / | / | 3.5-4 | 0 |
| | C | | | | | |
| 350 ppm | Acid cellulase | 120 | Catalase T | 1 | 3.5-4 | 0 |
| | C | | 100 | | | |
| 350 ppm | Acid cellulase | 120 | ASC 200 | 0.58 | 3.5-4 | 0 |
| | C | | | | | |

### Example 9

### Combining Biopolishing and Bleach Clean Up Into One Process Conducted with a Laundry-meter at Lower Temp

A 100% 460-60 bleached interlock cotton fabric was purchased from Test Fabrics. Fabric swatches were cut to about 5 g each.

One buffer at pH 6.5 was used for this study. 2.716 g of potassium dihydrogen phosphate and 0.201 g sodium hydroxide were dissolved in 1 L de-ionized water. The process was conducted with a Laundry-meter. The beaker was filled with 100 ml buffer and two pieces of pre-cut fabric.

1) Main washing: The beaker was filled with 100 ml buffer. Some hydrogen peroxide was added to each beaker with 20 steel balls as specified. In the meanwhile, a neutral Cellulase B was dosed to a concentration and catalase A was added in then temperature was raised to 30° C. and kept for 60 min.
2) Check the residual Hydroperoxide with peroxide strip.
3) Inactivation: After the checking, add the 1 g/l Na₂CO₃ in the machine/beaker then raised the temperature to 80° C. and run for 10 min, drained.
4) Cold rinse: Filled in cold water and rinsed for 10 min
5) Spin off the water on the fabrics and tumble dryer.
6) Measure the weight loss and pilling result on the treated fabrics.

The results of the test are shown in Table 6.

TABLE 6

| | | | | | | Residual |
|---|---|---|---|---|---|---|
| H₂O₂ | | Dosage | | Dosage | Pilling | H₂O₂ after |
| At beginning | Cellulase | (mg protein/l) | Catalase | (mg protein/l) | result | treatment |
| / | / | / | / | / | 1.5-2 | 0 |
| / | Neutral | 6 | / | / | 4 | 0 |
| | cellulase B | | | | | |
| 350 ppm | Neutral | 6 | Catalase | 0.34 | 4 | 0 |
| | cellulase B | | A | | | |
| / | Neutral | 12 | / | / | 4-4.5 | 0 |
| | cellulase B | | | | | |
| 350 ppm | Neutral | 12 | Catalase | 0.34 | 4.5 | 0 |
| | cellulase B | | A | | | |
| 350 ppm | Neutral | 7 | Catalase | 0.34 | 4 | 0 |
| | cellulase A | | A | | | |

### Example 10

### Combining Biopolishing and Bleach Clean Up with Reducing Agent Into One Process Conducted with a Laundry-Meter

A 100% 460-60 bleached interlock cotton fabric was purchased from Test Fabrics. Fabric swatches were cut to about 5 g each.

One buffer at pH 6.5 was used for this study. 2.716 g of potassium dihydrogen phosphate and 0.201 g sodium hydroxide were dissolved in 1 L de-ionized water. The process was conducted with a Laundry-meter. The beaker was filled with 100 ml buffer and two pieces of pre-cut fabric.

1) Main washing: The beaker was filled with 100 ml buffer. Some hydrogen peroxide was added to each beaker with 20 steel balls as specified. In the meanwhile, a neutral Cellulase B was dosed to a concentration and reducing agent (sodium hyposulphate: abbreviated as Hypo) was added in then temperature was raised to 55° C. and kept for 60 min.
2) Check the residual Hydroperoxide with peroxide strip.
3) Inactivation: After the checking, add the 1 g/l Na₂CO₃ in the machine/beaker then raised the temperature to 80° C. and run for 10 min, drained.
4) Cold rinse: Filled in cold water and rinsed for 10 min
5) Spin off the water on the fabrics and tumble dryer.
6) Measure the weight loss and pilling result on the treated fabrics.

The results of the test are shown in Table 7.

TABLE 7

| H₂O₂ | | Dosage | Reducing | Weight | Residual | Pilling |
|---|---|---|---|---|---|---|
| At beginning | Enzyme | (mg protein/l) | agent | loss % | H₂O₂ | result |
| / | | Blank | / | −0.6 | 0 | 1.625 |
| H₂O₂ 350 | Neutral | 3 | / | 2.9 | >25 ppm | 4.875 |
| ppm | Cellulase B | | | | | |
| H₂O₂ 350 | Neutral | 6 | / | 4.3 | >25 ppm | 5 |
| ppm | Cellulase B | | | | | |
| H₂O₂ 350 | Neutral | 12 | / | 6.5 | >25 ppm | 5 |
| ppm | Cellulase B | | | | | |
| H₂O₂ 100 | Neutral | 3 | 1 g/l | 3.4 | 0 | 5 |
| ppm | Cellulase B | | Hypo | | | |
| H₂O₂ 100 | Neutral | 6 | 1 g/l | 4.9 | 0 | 5 |
| ppm | Cellulase B | | Hypo | | | |
| H₂O₂ 100 | Neutral | 12 | 1 g/l | 6.6 | 0 | 5 |
| ppm | Cellulase B | | Hypo | | | |

### Example 11

### One Bath BioPolishing. Bleach Clean Up and Dyeing Conducted with a Laundry-Meter

A 100% 460-60 bleached interlock cotton fabric was purchased from Test Fabrics. Fabric swatches were cut to about 5 g each.

TABLE 8
Dyeing recipe

| | Sample No. | | | | | | | | | | | |
|---|---|---|---|---|---|---|---|---|---|---|---|---|
| | Sample 1 | | Sample 2 | | Sample 3 | | Sample 4 | | Sample 5 | | Sample 6 | |
| Chemicals used | Dosage | | | | | | | | | | | |
| H₂O₂ | 300 | ppm | / | | 300 | ppm | / | | 300 | ppm | / | |
| Neutral Cellulase | 7 | mg/L | / | | 7 | mg/L | / | | 7 | mg/L | / | |
| A(protein/L) | | | | | | | | | | | | |
| Catalase A(protein/L) | 0.34 | mg/l | | | 0.34 | mg/l | | | 0.34 | mg/l | | |
| Reactive RED BF-3B | 1% | owf | 1% | owf | 2% | owf | 2% | owf | 3% | owf | 3% | owf |
| Reactive Yellow BF-3R | 1% | owf | 1% | owf | 2% | owf | 2% | owf | 3% | owf | 3% | owf |
| Reactive TQ Blue G | 1% | owf | 1% | owf | 2% | owf | 2% | owf | 3% | owf | 3% | owf |
| Na₂SO₄ | 40 | g/l | 40 | g/l | 60 | g/l | 60 | g/l | 80 | g/l | 80 | g/l |
| Na₂CO₃ | 15 | g/l | 15 | g/l | 20 | g/l | 20 | g/l | 20 | g/l | 20 | g/l |

Owf: of the weight of fabric

A water solution at pH 6.5 was used for this study. Acetic acid was dissolved in 1 L de-ionized water to adjust pH to 6.5. The process was conducted with a Laundry-meter. The beaker was filled with 100 ml solution and two pieces of pre-cut fabric.

1) Single-bath bleach clean up, bio-polishing and dyeing process: The beaker was filled with 100 ml water solution at pH 6.5 adjusted by acetic acid. Some hydrogen peroxide was added to each beaker with 20 steel balls as specified. In the meanwhile, a neutral Cellulase A was dosed to a concentration and catalase A was added, following that certain amount of sodium sulfate was added in, then temperature was raised to 60° C. and kept for 15 min. Check the residual Hydroperoxide with peroxide strip.
2) Adding dyestuff and sodium carbonate: After the checking, add certain concentration of dyestuffs (three kinds of dyestuff from Argus) in the machine/beaker and run for 45 min, then dosed certain amount sodium carbonate and run for 45 min/60 min. Drained.
3) Soaping: two steps of soaping process were conducted following the dyeing process. Filled in cold water and added 1 g/l soaping agent Dekol SNS (BASF) and raised temperature to 90° C. for 10 min. Drained. Filled in cold water and added 1 g/l soaping agent Dekol SNS (BASF) and raised temperature to 90° C. for 10 min. Drained.
4) Hot rinse: Filled in cold water and raised temperature to 70° C. for 15 min.
5) Cold rinse: Filled in cold water and rinsed for 10 min.
6) Spin off the water on the fabrics and tumble dryer.
7) Measure the pilling result on the treated fabrics.

The results of the test are shown in Table 9.

TABLE 9

| | Sample No | | | | | |
|---|---|---|---|---|---|---|
| | Sample 1 | Sample 2 | Sample 3 | Sample 4 | Sample 5 | Sample 6 |
| Residual H₂O₂ before | 0 | n/a | 0 | n/a | 0 | n/a |
| adding dyestuff | | | | | | |
| Pilling | 4.5 | 1.5 | 4.25 | 1.5 | 4.25 | 1.5 |

As can be seen from the above table, the fabrics treated in the one bath bleach clean up, bio-polishing and dyeing process of the invention with a combination of catalase and cellulase have less pilling and largely shorten the process time compared to the separated process.

All patents, patent applications, and literature references referred to herein are hereby incorporated by reference in their entirety. Many variations of the present invention will suggest themselves to those skilled in the art in light of the above detailed description. Such obvious variations are within the full-intended scope of the appended claims.

1-32. (canceled)

33. A one-step process for combined biopolishing and bleach clean up of treating textile, comprising the step of treating the textile with (i) a system for removing hydrogen peroxide, and (ii) an enzyme system for bio-polishing, wherein the system for removing hydrogen peroxide and the enzyme system for bio-polishing are added simultaneously or sequentially to a single solution containing the textile.

34. The process of claim 33, wherein the system for removing hydrogen peroxide comprises a catalase or chemical reducing agent.

35. The process of claim 34, wherein the catalase is derived from bacterial, yeast, fungi, or animal.

36. The process of claim 35, wherein the catalase is derived from bacteria such as Bacillus, Pseudomonas or Streptomyces strain; yeast such as Candida, Kluyveromyces, Pichia, Saccharomyces, Schizosaccharomyces or Yarrowia; fungal such as Acremonium, Aspergillus, Aureobasidium, Bjerkandera, Cenporiopsis, Coprinus, Coriolus, Cryptococcus, Filibasidium, Fusarum, Humicola, Magnaporthe, Micrococcus Mucor, Myceliphthora, Neocallimastix, Neurospora, Paecilomyces, Penicillium, Phanerochaete, Phlebia, Piromyces, Pleurotus, Schizophyllum, Scytalidium, Talaromyces, Thermoascus, Thielavia, Tolypocladium, Trametes, or Trichoderma strain; or animal such as pig liver, beef lever.

37. The process of claim 34, wherein the catalase is Scytalidium thermophilum catalase of SEQ ID NO: 3, or a variant thereof.

38. The process of claim 33, wherein the enzyme system for biopolishing comprises a cellulase.

39. The process of claim 38, wherein the cellulase is derived from species of Aspergillus, Bacillus, Fusarium, Geotricum, Humicola, Irpex, Myceliophthora, Penicillium, Phanerochaete, Schizophyllum, Scytalidium, Thermomyces, Thielavia, or Trichoderma.

40. The process of claim 39, wherein the cellulase is derived from Aspergillus aculeatus, Aspergillus niger, Humicola insolens, Myceliophthora thermophila, Trichoderma harzianum, Trichoderma reesei, or Thielavia terrestis.

41. The process of claim 38, wherein the cellulase is a family 45 cellulase.

42. The process of claim 38, wherein the cellulase is selected from the group consisting of a polypeptide comprising the sequence of SEQ ID NO: 1 or SEQ ID NO. 2, or a variant thereof.

43. The process of claim 33, wherein the enzyme system for biopolishing comprises a protease.

44. The process of claim 2, wherein the reducing agent is sodium hyposulphate.

45. A process for combined bleach clean up, biopolishing and dyeing of treating textile in a single bath, comprising
(i) adding into the aqueous solution an enzyme system for removing hydrogen peroxide,
(ii) adding an enzyme system for bio-polishing, wherein steps (i) and (ii) take place simultaneously or sequentially in a single solution containing the textile, and
(iii) supplementing the aqueous solution with one or more dyestuff and incubating the textile for a sufficient time to achieve dyed textile.

46. An enzyme composition comprising (i) a system for removing hydrogen peroxide and (ii) an enzyme system for bio-polishing.


SEQUENCE LISTING (3 sequences)

SEQ ID NO: 1 - protein, 299 aa, Thielavia terrestis
MRSTPVLRTTLAAALPLVASAASGSGQSTRYWDCCKPSCAWPGKAAVSQPVYACDANFQRLSDFNVQSGCNGGSAYSCADQTPWAVNDNLAYGFAATSIAGGSESSWCCACYALTFTSGPVAGKTMVVQSTSTGGDLGSNQFDIAMPGGGVGIFNGCSSQFGGLPGAQYGGISSRDQCDSFPAPLKPGCQWRFDWFQNADNPTFTFQQVQCPAEIVARSGCKRNDDSSFPVFTPPSGGNGGTGTPTSTAPGSGQTSPGGGSGCTSQKWAQCGGIGFSGCTTCVSGTTCQKLNDYYSQCL

SEQ ID NO: 2 - protein, 305 aa, Humicola insolens
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 3 - protein, 717 aa, Scytalidium thermophilum
MNRVTNLLAWAGAIGLAQATCPFADPAALYSRQDTTSGQSPLAAYEVDDSTGYLTSDVGGPIQDQTSLKAGIRGPTLLEDFMFRQKIQHFDHERVPERAVHARGAGAHGTFTSYADWSNITAASFLNATGKQTPVFVRFSTVAGSRGSADTARDVHGFATRFYTDEGNFDIVGNNIPVFFIQDAIQFPDLIHSVKPRPDNEIPQAATAHDSAWDFFSQQPSTMHTLFWAMSGHGIPRSYRHMDGFGVHTFRFVKDDGSSKLIKWHFKSRQGKASLVWEEAQVLSGKNADFHRQDLWDAIESGNGPEWDVCVQIVDESQAQAFGFDLLDPTKIIPEEYAPLTKLGLLKLDRNPTNYFAETEQVMFQPGHIVRGIDFTEDPLLQGRLFSYLDTQLNRNGGPNFEQLPINMPRVPIHNNNRDGAGQMFIHRNKYPYTPNTLNSGYPRQANQNAGRGFFTAPGRTASGALVREVSPTFNDHWSQPRLFFNSLTPVEQQFLVNAMRFEISLVKSEEVKKNVLTQLNRVSHDVAVRVAAAIGLGAPDADDTYYHNNKTAGVSIVGSGPLPTIKTLRVGILATTSESSALDQAAQLRTRLEKDGLVVTVVAETLREGVDQTYSTADATGFDGVVVVDGAAALFASTASSPLFPTGRPLQIFVDAYRWGKPVGVCGGKSSEVLDAADVPEDGDGVYSEESVDMFVEEFEKGLATFRFTDRFALDS